(19) United States
(12) Patent Application Publication
(10) Pub. No.: US 2010/0267828 A1
(43) Pub. Date: Oct. 21, 2010

(54) DHA DERIVATIVES AND THEIR USE AS MEDICAMENTS

(76) Inventors: Anne Kristin Holmeide, Oslo (NO); Ragnar Hovland, Nesoddtangen (NO)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/740,377

(22) PCT Filed: Oct. 30, 2008
(86) PCT No.: PCT/IB08/03666

§ 371 Date: Jul. 1, 2010

Related U.S. Application Data
(60) Provisional application No. 61/001,097, filed on Oct. 31, 2007.

(51) Int. Cl. A61K 31/232; C07C 69/587; C07C 67/00; C12P 7/64; A61P 3/10; A61P 3/04; A61P 29/00

(52) U.S. Cl. 514/549; 554/224; 554/219; 554/149; 435/134

(57) ABSTRACT

The present disclosure relates to compounds of the general formula (I):

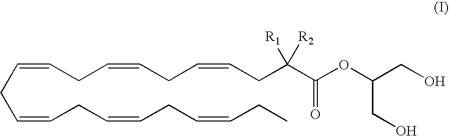

wherein
R₁ and R₂ are the same or different and are chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, an alkylsulfinyl group, an alkylsulfonyl group, an amino group, and an alkylamino group;
or any pharmaceutically acceptable salt, solvate, complex, or pro-drug thereof;
and their use as medicaments for the treatment of various diseases and conditions, for example, reduction of elevated triglyceride levels, i.e., hypertriglyceridemia, reduction of non-HDL cholesterol, reduction of glucose and HbA1c, and improvement of insulin resistance. The present disclosure also relates to a pharmaceutical composition comprising compounds of formula (I), as well as to processes for preparing compounds according to formula (I).

### TECHNICAL FIELD

The present disclosure relates to compounds of the general formula (I):

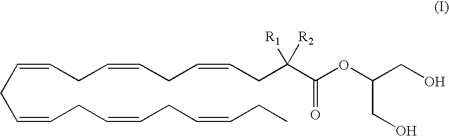

wherein
R₁ and R₂ are the same or different and are chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, an alkylsulfinyl group, an alkylsulfonyl group, an amino group, and an alkylamino group;

and any pharmaceutically acceptable salt, solvate, complex, or pro-drug thereof; and their use as medicaments for the treatment of various diseases and conditions, for example, reduction of elevated triglyceride levels, i.e., hypertriglyceridemia, reduction of non-HDL cholesterol, reduction of glucose and HbA1c, and improvement of insulin resistance. The present disclosure also relates to a pharmaceutical composition comprising compounds of formula (I), as well as to processes for preparing compounds according to formula (I).

### BACKGROUND OF THE INVENTION

EPA and DHA have effects on diverse physiological processes impacting normal health and chronic disease, such as the regulation of plasma lipid levels, cardiovascular and immune function, insulin action, neural development, and visual function. Firm evidence exists for their beneficial role in the prevention and management of coronary heart disease, dyslipidemias, type 2 diabetes, insulin resistance, and hypertension (Simonopoulos, A. P. Am. J. Clin. Nutr. (1999) 70(Suppl):560S-569S; Geleijnse, J. M. et al. J. Hypertension (2002) 20:1493-1499; Storlien, L. H. et al. Curr. Opin. Clin. Nutr. Metab. Care (1998) 1:559-563).

One such form of omega-3 fatty acids is a concentrate of omega-3, long chain, polyunsaturated fatty acids from fish oil containing DHA and EPA as ethyl esters, described, for example, in U.S. Pat. Nos. 5,502,077; 5,656,667; and 5,698,594, and is sold under the trademark Omacor® or Lovaza®. Specifically, a fatty acid composition containing a high concentration, of at least 80% by weight, of omega-3 fatty acids as ethyl esters, where EPA ethyl ester and DHA ethyl ester are present in relative amounts of 1:2 to 2:1, and constitute about at least 75% of the total fatty acids in the composition, has shown advantageous effects on several risk factors for cardiovascular diseases, especially exhibiting beneficial effects on hypertriglyceridemia, mild hypertension, and on the coagulation factor VII phospholipid complex activity. Such compounds, including Omacor® and Lovaza®, lower serum LDL-cholesterol, increase serum HDL-cholesterol, lower serum triglycerides, lower systolic and diastolic blood pressure and the pulse rate, and lower the activity of the blood coagulation factor VII-phospholipid complex. EPA and DHA have been shown to operate synergistically. Additionally, EPA and DHA, as well as the compounds according to formula (I) disclosed herein are very well tolerated and do not give rise to any severe side effects.

The increasing incidence of type 2 diabetes mellitus and cardiovascular diseases worldwide poses an immense public health and medical challenge for the implementation of successful preventive and treatment strategies. The concurrent rise in overweight people and high incidence of obesity, which is correlated to type 2 diabetes, interferes with diabetes treatment and increases the likelihood of hypertension, dyslipidemia, and atherosclerosis related diseases.

The pathophysiologic condition preceding the development of type 2 diabetes is related to reduced effects of insulin on peripheral tissues, called insulin resistance. These tissues are mainly muscle, fat, and liver. Muscle tissue is the main tissue affected by insulin resistance in type 2 diabetes. The syndrome characterized by insulin resistance, hypertension, dyslipidemia, and a systemic proinflammatory state, is referred to as metabolic syndrome. The prevalence of metabolic syndrome in the adult population in developed countries is 22-39% (Meighs, J. B. et al. Diabetes (2003) 52:2160-2167).

Currently the most promising approach to mitigate and deter metabolic syndrome is lifestyle intervention with weight reduction, decreased consumption of saturated fat, and increased physical activity in combination with appropriate pharmacotherapy. Healthy diets that avoid excess energy intake encompass substitution of mono- and polyunsaturated fatty acids in exchange for saturated fat. In particular the long-chain omega-3 fatty acids from fatty fish, namely eicosapentaenoic acid (EPA) and docosahexaenoic acid (DHA), have proven beneficial in prevention of type 2 diabetes.

Recent studies suggest that omega-3 fatty acids serve as important mediators of gene expression, working via nuclear receptors like the peroxisome proliferator-activated receptors (PPARs) controlling the expression of the genes involved in the lipid and glucose metabolism and adipogenesis (Jump, D. B. J. Biol. Chem. (2002) 277:8755-8758). PPARs are nuclear fatty acid receptors that have been implicated to play an important role in obesity-related metabolic diseases such as hyperlipidemia, insulin resistance, and coronary heart disease.

Recently, pharmaceuticals acting as ligands to the PPARγ receptor have been introduced for the treatment of type 2 diabetes (Yki-Jarvinen, H. New Engl. J. Med. (2004) 351:1106-1118).

However, these pharmaceuticals are generally accompanied by weight gain and an increase in the subcutaneous adipose-tissue mass (Adams, M. et al. J. Clin. Invest. (1997) 100:3149-3153). The use of thiazolidinediones is not only associated with weight gain but a subgroup of patients also have fluid retention and plasma volume expansion, leading to peripheral edema. The increase in body weight and edema has been associated with an increase in the incidence of heart failure, which is the reason why the Food and Drug Administration (FDA) has included a warning in the prescription information for rosiglitazone (provided by Avandia) and pioglitazone (provided by Takeda). These adverse effects restrict the use of the thioglitazones especially in patients with coronary heart conditions. Accordingly, there is a potential for new drugs with positive effects on insulin resistance but without weight reduction activity and no fluid retention tendency.

Although omega-3 fatty acids are weak agonists of PPARs, when compared with pharmacological agonists like thioglitazones, these fatty acids have demonstrated improvement in glucose uptake and insulin sensitivity (Storlien, L. H. et al. Science (1987) 237:885-888). It has been reported that adipocytes were more insulin sensitive and transported more glucose when the polyunsaturated to saturated fatty acid ratio in the diet was increased (Field, C. J. et al. J. Biol. Chem. (1990) 265:11143-11150). Collectively, these data indicate that the 20- and 22-carbon fatty acids, such as EPA and DHA, could play a preventive role in the development of insulin resistance.

Due to their limited stability in vivo and their lack of biological specificity, PUFAs have not achieved widespread use as therapeutic agents. Chemical modifications of the n-3 polyunsaturated fatty acids have been performed by several research groups in order to change or increase their metabolic effects.

For example, the hypolipidemic effects of EPA were potentiated by introducing methyl or ethyl in the α- or β-position of EPA. (Vaagenes, H. et al. Biochem. Pharmacol. (1999) 58:1133-1143). The compounds also reduced plasma free fatty acid while EPA ethyl ester (EE) had no effect.

In a work published by L. Larsen (Larsen, L. et al. Lipids (2005) 40:49-57), the authors show that the α-methyl derivatives of EPA and DHA increased the activation of the nuclear receptor PPARα and thereby the expression of L-FABP compared to EPA/DHA. EPA, having an ethyl group in the α-position, activated PPARα with equal strength as α-methyl EPA. The authors suggest that delayed catabolism of these α-methyl fatty acids may contribute to their increased effects due to decreased β-oxidation in mitochondria leading to peroxisomal oxidation.

Alpha-methyl EPA has been shown to be a stronger inhibitor of platelet aggregation than EPA, both in vitro (Larsen, L. et al. Biochemical Pharmacology (1998) 55:405) and in vivo (Willumsen, N. Biochim Biophys Acta (1998) 1369:193-203).

Patent Abstract of Japan, publication number 05-00974 discloses DHA substituted in the alpha-position with an OH-group, however only as an intermediate. No mention of possible pharmaceutical effects of this compound is disclosed.

Laxdale Limited has also described the use of alpha substituted derivatives of EPA in the treatment of psychiatric or central nervous disorders (U.S. Pat. No. 6,689,812).

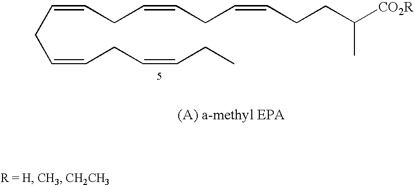

None of those modified fatty acids have, however, shown satisfactory pharmaceutical activity, and none of them has reached the pharmaceutical market.

Reference may also be made to WO 2006/117664, which discloses DHA derivatives having alpha-substitution.

### SUMMARY OF THE INVENTION

The aim of the present disclosure is to provide new DHA-derivatives having therapeutic activity.

Accordingly, the present disclosure relates to a compound of formula (I):

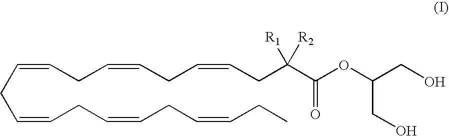

wherein
R₁ and R₂ are the same or different and are chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, an alkylsulfinyl group, an alkylsulfonyl group, an amino group, and an alkylamino group;
   and any pharmaceutically acceptable salts, solvates, complexes, and pro-drugs thereof

In at least one embodiment, the alkyl group is chosen from methyl, ethyl, n-propyl, isopropyl, n-butyl, iso-butyl, sec-butyl, n-hexyl, and benzyl.

In at least one embodiment, the halogen atom is chosen from fluorine, chlorine, bromine, and iodine.

In at least one embodiment, the alkoxy group is chosen from methoxy, ethoxy, propoxy, isopropoxy, sec-butoxy, phenoxy, benzyloxy, OCH₂CF₃, and OCH₂CH₂OCH₃.

In at least one embodiment, the alkenyl group is chosen from allyl, 2-butenyl, and 3-hexenyl.

In at least one embodiment, the alkynyl group is chosen from propargyl, 2-butynyl, and 3-hexynyl.

In at least one embodiment, the aryl group is a phenyl group.

In at least one embodiment, the alkylthio group is chosen from methylthio, ethylthio, isopropylthio, and phenylthio.

In at least one embodiment, the alkoxycarbonyl group is chosen from methoxycarbonyl, ethoxycarbonyl, propoxycarbonyl, and butoxycarbonyl.

In at least one embodiment, the alkylsulfinyl group is chosen from methanesulfinyl, ethanesulfinyl, and isopropanesulfinyl.

In at least one embodiment, the alkylsufinyl group is chosen from methanesulfonyl, ethanesulfonyl, and isopropanesulfonyl.

In at least one embodiment, the alkylamino group is chosen from methylamino, dimethylamino, ethylamino, and diethylamino.

In at least one embodiment, R₁ and R₂ are chosen from a hydrogen atom, a hydroxy group, an alkyl group, a halogen atom, an alkoxy group, an alkylthio group, an alkylsulfinyl group, an alkylsulfonyl group, an amino group, and an alkylamino group.

In at least one embodiment, R₁ and R₂ are chosen from a hydrogen atom, a hydroxy group, a C₁-C₇ alkyl group, a halogen atom, a C₁-C₇ alkoxy group, a C₁-C₇ alkylthio group, a C₁-C₇ alkylsulfinyl group, a C₁-C₇ alkylsulfonyl group, an amino group, and a C₁-C₇ alkylamino group.

In at least one embodiment, the C₁-C₇ alkyl group is methyl, ethyl, or benzyl; the halogen atom is fluorine or iodine; the C₁-C₇ alkoxy group is methoxy or ethoxy; the C₁-C₇ alkylthio group is methylthio, ethylthio, or phenylthio; the C₁-C₇ alkylsulfinyl group is ethanesulfinyl; the C₁-C₇ alkylsulfonyl group is ethanesulfonyl; the C₁-C₇ alkylamino group is ethylamino or diethylamino.

In at least one embodiment, R₁ and R₂ are chosen from a hydrogen atom, a C₂-C₇ alkyl group, a halogen atom, a C₁-C₇ alkoxy group, a C₁-C₇ alkylthio group, a C₁-C₇ alkylsulfinyl group, a C₁-C₇ alkylsulfonyl group, an amino group, and a C₁-C₇ alkylamino group.

In at least one embodiment, the C₂-C₇ alkyl group is ethyl or benzyl; the halogen atom is fluorine or iodine; the C₁-C₇ alkoxy group is methoxy or ethoxy; the C₁-C₇ alkylthio group is methylthio, ethylthio, or phenylthio; the C₁-C₇ alkylsulfinyl group is ethanesulfinyl; the C₁-C₇ alkylsulfonyl group is ethanesulfonyl; the C₁-C₇ alkylamino group is ethylamino or diethylamino.

In the compounds according to formula (I) of the present disclosure, R₁ and R₂ may be the same or different. When they are different, the compounds of formula (I) are capable of existing in stereoisomeric forms. It will be understood that the present disclosure encompasses all optical isomers of the compounds of formula (I) and mixtures thereof including racemates.

The present disclosure includes where R₁ is different from R₂ compounds of formula (I) that are racemic or enantiomerically enriched, or enantiomerically pure, either as the (S) or (R) enantiomer.

In at least one embodiment of the present disclosure, the use of a compound according to formula (I) is disclosed for the manufacture of a medicament for treating or preventing various conditions, such as for controlling body weight reduction and/or for preventing body weight gain, for the treatment and/or the prevention of obesity or an overweight condition, for the prevention and/or treatment of diabetes (e.g., type 2 diabetes), for the treatment and/or prevention of amyloidos-related diseases, for the treatment or prophylaxis of multiple risk factors for cardiovascular diseases, for the treatment of elevated blood lipids, such as triglycerides and non-HDL cholesterol (e.g., LDL cholesterol and VLDL cholesterol), for the treatment of elevated insulin levels, for the treatment of dyslipidemia, for the treatment of hypertension, post-myocardial infarction (MI), depression, and IgA nephropathy, for increasing serum HDL levels, for the treatment and/or prevention of an inflammatory disease or condition, and/or for prevention of stroke, cerebral or transient ischemic attacks related to atherosclerosis of several arteries.

In at least one embodiment of the present disclosure, the compound of formula (I) is a compound of formula (Ia):

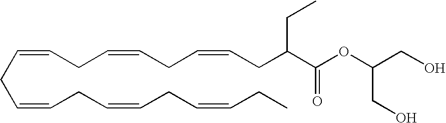

### (Ia) (all-Z)-1,3-dihydroxypropan-2-yl 2-ethyl-docosa-4,7,10,13,16,19-hexaenoate

or any pharmaceutically acceptable salt, solvate, complex, or pro-drug thereof. Also included within the scope of the present disclosure are methods for preparing a compound of formula (Ia).

The alpha-substituted DHA-derivatives, such as monoglycerides, according to the present disclosure have shown pharmaceutical activity. In particular, the fatty acid derivatives, such as monoglycerides, according to the present disclosure may be capable of being used in the treatment and/or prevention of those diseases and conditions disclosed herein.

Another aspect of the present disclosure relates to a method of using a compound of formula (I) as a medicament.

The present disclosure also relates to a process for the manufacture of a compound of formula (I). For example, a compound of formula (I) may be prepared from (all-Z)-4,7,10,13,16,19-docosahexaenoic acid (DHA). The DHA may originate, for example, from a vegetable, a microbial, and/or an animal source, such as a marine oil, for example fish oil. Another advantage of a compound of formula (I) is that the fatty acid compounds may be prepared directly from (all-Z)-4,7,10,13,16,19-docosahexaenoic acid (DHA).

In an embodiment of the present disclosure, the compounds of formula (I) are prepared from DHA, wherein the DHA is obtained from at least one origin chosen from vegetable origin, microbial origin, and animal origin. The present disclosure also includes derivatives prepared from DHA-containing oil from microbial origin. Suitable DHA may also be obtained from a marine oil, such as a fish oil.

Another aspect of the present disclosure relates to a pharmaceutical composition comprising a compound of formula (I) as an active ingredient. The pharmaceutical composition may further comprise a pharmaceutically acceptable carrier. Suitably, a pharmaceutical composition according to the present disclosure is formulated for oral administration, e.g., in the form of a capsule or a sachet. A suitable daily dosage of a compound of formula (I) according to the present disclosure ranges from 10 mg to 10 g, such as from 100 mg to 1 g of the compound of formula (I).

In addition, the present disclosure relates to a fatty acid composition comprising a compound of formula (I), wherein the compound of formula (I) may be present in an at least 60%, such as at least 90%, by weight of the fatty acid composition. The fatty acids may be present in the form of derivatives. A fatty acid composition according to the present disclosure may further comprise a pharmaceutically acceptable antioxidant, e.g., tocopherol. Within the scope of the present disclosure is also a fatty acid composition described above for use as a medicament.

In a further aspect, the present disclosure relates to the use of a compound according to formula (I) for the manufacture of a medicament for controlling body weight reduction and/or for preventing body weight gain; for the manufacture of a medicament for the treatment and/or the prevention of obesity or an overweight condition; for the manufacture of a medicament for the prevention and/or treatment of diabetes, such as type 2 diabetes; for the manufacture of a medicament for the treatment and/or prevention of amyloidos-related diseases; for the manufacture of a medicament for the treatment or prophylaxis of multiple risk factors for cardiovascular diseases, such as for the treatment of elevated blood lipids; and for the manufacture of a medicament for prevention of stroke, cerebral or transient ischemic attacks related to atherosclerosis of several arteries.

In addition, the present disclosure relates to a method for controlling body weight reduction and/or for preventing body weight gain; a method for the treatment and/or the prevention of obesity or an overweight condition; a method for the prevention and/or treatment of diabetes, such as type 2 diabetes; a method for the treatment and/or prevention of amyloidos-related diseases; a method for the treatment or prophylaxis of multiple risk factors for cardiovascular diseases; and a method for the prevention of stroke, cerebral or transient ischemic attacks related to atherosclerosis of several arteries, wherein a pharmaceutically effective amount of a compound of formula (I) is administered to a human or an animal. Suitably, the compound of formula (I) is administered orally to a human or an animal.

### BRIEF DESCRIPTION OF THE DRAWINGS

FIG. 1 is a schematic of the plasma triglyceride levels measured in Test Example 1.

FIG. 2 is a schematic of the plasma cholesterol levels measured in Test Example 1.

FIG. 3 depicts the receptor activity measured in Test Example 2.

### DETAILED DESCRIPTION OF THE INVENTION

The inventors have found that monoglycerides of alpha-substituted DHA derivatives have potent lipid lowering effects in animal models. The compounds also have affinity to the nuclear receptors, PPARα and PPARγ. Because of this, they will have a potential as pharmaceuticals for the treatment of various diseases and conditions.

Throughout this description, the abbreviation “PRB-x MG,” where x is an integer, will be used when describing exemplary compounds according to the present disclosure.

Exemplary categories of compounds according to the present disclosure include the following:

Category A: R₁ is an Alkyl Group, and R₂ is a Hydrogen Atom.

### Example i

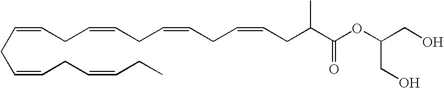

(all-Z)-1,3-dihydroxypropan-2-yl 2-methyl-docosa-4,7,10,13,16,19-hexaenoate (PRB-1 MG), wherein R₁ is a methyl group, and R₂ is a hydrogen atom.

### Example ii

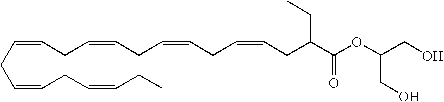

(all-Z)-1,3-dihydroxypropan-2-yl 2-ethyl-docosa-4,7,10,13,16,19-hexaenoate (PRB-2 MG) (Ia), wherein R₁ is an ethyl group, and R₂ is a hydrogen atom.

### Example iii

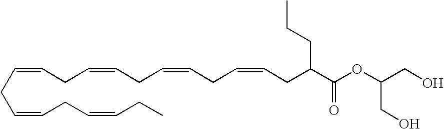

(all-Z)-1,3-dihydroxypropan-2-yl 2-propyl-docosa-4,7,10,13,16,19-hexaenoate (PRB-3 MG), wherein R₁ a is propyl group, and R₂ is a hydrogen atom.

Category B: R₁ and R₂ are Both Alkyl Groups

### Example iv

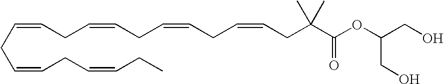

(all-Z)-1,3-dihydroxypropan-2-yl 2,2-dimethyl-docosa-4,7,10,13,16,19-hexaenoate (PRB-4 MG), wherein R₁ and R₂ are methyl groups.

Category C: R₁ is an Alkoxy Group, and R₂ is a Hydrogen Atom

### Example v

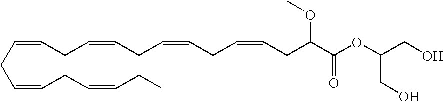

(all-Z)-1,3-dihydroxypropan-2-yl 2-methoxy-docosa-4,7,10,13,16,19-hexaenoate (PRB-5 MG), wherein R₁ is a methoxy group, and R₂ is a hydrogen atom.

### Example vi

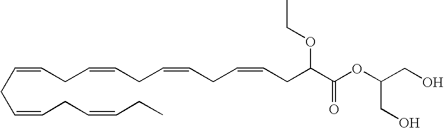 [0075] (all-Z)-1,3-dihydroxypropan-2-yl 2-ethoxy-docosa-4,7,10,13,16,19-hexaenoate (PRB-6 MG), wherein R₁ is an ethoxy group, and R₂ is a hydrogen atom.

It is to be understood that the present disclosure encompasses all pharmaceutically acceptable salts, solvates, complexes, and pro-drugs of compounds of formula (I).

“Pro-drugs” are entities which may or may not possess pharmacological activity as such, but may be administered (such as orally or parenterally) and thereafter subjected to bioactivation (for example metabolized) in the body to form the agent of the present disclosure which is pharmacologically active.

A “therapeutically effective amount” or a “pharmaceutically effective amount” relates to an amount that will lead to the desired therapeutic and/or pharmacological effects. While individual patient needs may vary, determination of optimal ranges for effective amounts of compounds of formula (I) is within the skill of the skilled artisan. Generally, the dosage regimen for treating a condition with the compounds and/or compositions of this present disclosure is chosen in accordance with a variety of factors, including the type, age, weight, sex, diet, and medical condition of the patient.

By “a medicament” is meant a compound according to formula (I), in any form suitable to be used for a medical purpose, e.g., in the form of a medicinal product, a pharmaceutical preparation or product, a dietary product, a food stuff, or a food supplement.

In the context of the present specification, the term “therapy” also includes “prophylaxis” and “prevention” unless there are specific indications to the contrary. The terms “therapeutic” and “therapeutically” should be constructed accordingly.

Treatment includes any therapeutic application that can benefit a human or non-human animal. The treatment of mammals is an exemplary embodiment. Both human and veterinary treatments are within the scope of the present disclosure. Treatment may be for an existing condition or it may be prophylactic, i.e., preventative. Treatment may be of an adult, a juvenile, an infant, a fetus, or a part of any of the aforesaid (e.g. an organ, tissue, cell, or nucleic acid molecule). By “chronic treatment” is meant treatment that continues for some weeks or years.

A compound according to the present disclosure may, for example, be included in a food stuff, a food supplement, a nutritional supplement, or a dietary product. Alpha-substituted DHA derivatives and EPA (or DHA for that matter) can be bound together and combined on triglycerides by an esterification process between a mixture of alpha-derivatives, EPA, and glycerol catalyzed by Novozym 435 (a commercially available lipase from Candida antarctica on immobilized form). The compounds of formula (I) have activity as pharmaceuticals, such as triggers of nuclear receptor activity.

In at least one embodiment, the novel compounds of formula (I), pharmaceutically acceptable salts, solvates, complexes, and/or pro-drugs thereof, may be used for controlling body weight reduction and/or for preventing body weight gain, for the treatment and/or the prevention of obesity or an overweight condition, for the prevention and/or treatment of diabetes (e.g., type 2 diabetes), for the treatment and/or prevention of amyloidos-related diseases, for the treatment or prophylaxis of multiple risk factors for cardiovascular diseases, for the treatment of elevated blood lipids, such as triglycerides and non-HDL cholesterol, for the treatment of elevated insulin levels, for the treatment of dyslipidemia, for the treatment of hypertension, post-myocardial infarction (MI), depression, and IgA nephropathy, for increasing serum HDL levels, for the treatment and/or prevention of inflammatory diseases or conditions, and/or for prevention of stroke, cerebral or transient ischemic attacks related to atherosclerosis of several arteries.

There are two major forms of diabetes mellitus. One is type 1 diabetes, which is known as insulin-dependent diabetes mellitus (IDDM), and the other one is type 2 diabetes, which is known as non-insulin-dependent diabetes mellitus (NIDDM). Type 2 diabetes is related to obesity/overweight and lack of exercise, often of gradual onset, usually in adults, and caused by reduced insulin sensitivity, namely peripheral insulin resistance. This leads to a compensatory increase in insulin production. This stage before developing full fledged type 2 diabetes is called the metabolic syndrome and is characterized by hyperinsulinemia, insulin resistance, obesity, glucose intolerance, hypertension, abnormal blood lipids, hypercoagulopathia, dyslipidemia, and inflammation, often leading to atherosclerosis of the arteries. Later when insulin production ceases, type 2 diabetes mellitus develops.

In an exemplary embodiment, the compounds according to formula (I) may used for the treatment of type 2 diabetes. The compounds according to formula (I) may also be used for the treatment of other types of diabetes chosen from metabolic syndrome, secondary diabetes, such as pancreatic, extrapancreatic/endocrine, or drug-induced diabetes, and exceptional forms of diabetes, such as lipoatrophic, myatonic, or a disease caused by disturbance of the insulin receptors.

In at least one embodiment, compounds of formula (I) may activate nuclear receptors, such as PPAR (peroxisome proliferator-activated receptor) α and/or γ.

The compounds of formula (I) may also be used for the treatment and/or prevention of obesity. Obesity is usually linked to an increased insulin resistance, and obese people run a high risk of developing type 2 diabetes, which is a major risk factor for the development of cardiovascular diseases. Obesity is a chronic disease that afflicts an increasing proportion of the population in Western societies and is associated, not only with a social stigma, but also with decreasing life span and numerous problems, for instance diabetes mellitus, insulin resistance, and hypertension.

The compounds according to formula (I) may also be used for the prevention and/or treatment of amyloidos-related diseases. Amyloidos-related conditions or diseases associated with deposition of amyloid, such as a consequence of fibril or plaque formation, include Alzheimer's disease or dementia, Parkinson's disease, amyotropic lateral sclerosis, the spongiform encephalopathies, such as Creutzfeld-Jacob disease, cystic fibrosis, primary or secondary renal amyloidoses, IgA nephropathy, and amyloid deposition in arteries, myocardium and neural tissue. The treatment of an amyloidos-related disease can be made either acutely or chronically.

The compounds of formula (I) may be administered to patients with symptoms of atherosclerosis of arteries supplying the brain, for instance a stroke or transient ischemic attack, in order to reduce the risk of a further possible fatal attack.

The compounds of formula (I) may also be used for the treatment of elevated blood lipids in humans.

Additionally, the compounds of formula (I) may also be used for the treatment and/or prophylaxis of multiple risk factors known for cardiovascular diseases, such as hypertension and hypertriglyceridemia. In an exemplary embodiment, the compounds of formula (I) are used for the treatment of elevated blood lipids in humans.

The compounds of formula (I), pharmaceutically acceptable salts, solvates, pro-drugs, and/or complexes thereof, may be used on their own but may also be administered in the form of a pharmaceutical composition in which the compounds of formula (I) (the active ingredient) are in association with a pharmaceutically acceptable excipient, diluent, or carrier (including combinations thereof).

The present disclosure also relates to a pharmaceutical composition comprising a therapeutically effective amount of the compound of formula (I) and a pharmaceutically acceptable carrier, diluent, or excipient (including combinations thereof).

Acceptable carriers, diluents, and excipients for therapeutic/pharmaceutical use are well known in the pharmaceutical art. The choice of pharmaceutical carrier, excipient, and/or diluent (including combinations thereof) can be selected with regard to the intended route of administration and standard pharmaceutical practice. The pharmaceutical compositions may comprise as the carrier, excipient, or diluent, or in addition thereto, any suitable binder(s), lubricant(s), suspending agent(s), coating agent(s), and solubilizing agent(s).

Pharmaceutical compositions within the scope of the present disclosure may include one or more of the following: preserving agents, solubilizing agents, stabilizing agents, wetting agents, emulsifiers, sweeteners, colorants, flavoring agents, odorants, salts (compounds of the present disclosure may themselves be provided in the form of a pharmaceutically acceptable salt), buffers, coating agents, antioxidants, suspending agents, adjuvants, excipients, and diluents.

In at least one embodiment, a pharmaceutical composition according to the present disclosure may be formulated for oral administration to a human or an animal. The pharmaceutical composition may also be formulated for administration through any other route where the active ingredients may be efficiently absorbed and utilized, e.g., intravenously, subcutaneously, intramuscularly, intranasally, rectally, vaginally, or topically.

In at least one embodiment of the present disclosure, the pharmaceutical composition is shaped in the form of a capsule, which could also be microcapsules generating a powder or a sachet. The capsule may be flavored. This embodiment also includes a capsule wherein both the capsule and the encapsulated fatty acid composition according to the present disclosure are flavored. By flavoring the capsule, it may be more attractive to the user. For the above-mentioned therapeutic uses the dosage administered will, of course, vary with the compound employed, the mode of administration, the treatment desired, and the disorder indicated.

In at least one embodiment, the pharmaceutical composition may be formulated to provide a daily dosage ranging from 10 mg to 10 g. In an exemplary embodiment, the pharmaceutical composition is formulated to provide a daily dosage ranging from 50 mg to 5 g of the composition. In another exemplary embodiment, the pharmaceutical composition is formulated to provide a daily dosage ranging from 100 mg to 1 g of the composition. By a daily dosage is meant the dosage per 24 hours. The dosage administered will, of course, vary with the compound employed, the mode of administration, the treatment desired, and the disorder indicated. A physician will determine the actual dosage which will be most suitable for an individual subject. The specific dose level and frequency of dosage for any particular patient may be varied and will depend upon a variety of factors, including the activity of the compound of formula (I) employed, the metabolic stability and length of action of that compound, the age, body weight, general health, sex, diet, mode and time of administration, rate of excretion, drug combination, the severity of the particular condition, and the individual undergoing therapy. The agent and/or the pharmaceutical composition of the present disclosure may be administered from 1 to 10 times per day, such as once or twice per day. For oral and parenteral administration to human patients, the daily dosage level of the agent may be in single or divided doses.

Another embodiment of the present disclosure relates to a fatty acid composition comprising compounds of formula (I).

The fatty acid composition may comprise in the range of 60% to 100% by weight of the compounds of formula (I), all percentages by weight being based on the total weight of the fatty acid composition. In an exemplary embodiment of the present disclosure, the compounds of formula (I) are present in an amount of at least 80%, such as 90%, such as 95% by weight of the fatty acid composition.

The fatty acid composition according to the present disclosure may comprise (all-Z omega-3)-6,9,12,15,18-heneicosapentaenoic acid (HPA) or derivatives thereof in an amount of at least 1% by weight, or in an amount of 1% to 4% by weight.

The fatty acid composition according to the present disclosure may comprise omega-3 fatty acids other than EPA and DHA that have 20-, 21-, or 22-carbon atoms, or derivatives thereof, in an amount of at least 1.5% by weight, or in an amount of at least 3% by weight.

In at least one embodiment of the present disclosure, the fatty acid composition is a pharmaceutical composition, a nutritional composition, or a dietary composition. The fatty acid composition may further comprise an effective amount of a pharmaceutically acceptable antioxidant, such as tocopherol or a mixture of tocopherols. In an exemplary embodiment, the fatty acid composition further comprises tocopherol, or a mixture of tocopherols, in an amount of up to 4 mg per g of the total weight of the fatty acid composition. In at least one embodiment, the fatty acid composition comprises an amount of 0.2 mg to 0.4 mg per g of tocopherols, based on the total weight of the composition.

Another aspect of the present disclosure provides a fatty acid composition, or any pharmaceutically acceptable salt, solvate, pro-drug, or complex thereof, comprising compounds of formula (I) for use as a medicament and/or in therapy. Such a fatty acid composition may be used to prevent and/or treat the same conditions as outlined herein for the compounds of formula (I).

When the fatty acid composition is used as a medicament, it may be administered in a therapeutically or a pharmaceutically active amount.

In an embodiment, the fatty acid composition is administered orally to a human or an animal.

The present disclosure also provides for the use of a compound of formula (I), a pharmaceutically acceptable salt, solvate, pro-drug, and/or complex thereof for the manufacture of a medicament for controlling body weight reduction and/or for preventing body weight gain; for the manufacture of a medicament for the treatment and/or the prevention of obesity or an overweight condition; for the manufacture of a medicament for the prevention and/or treatment of diabetes; for the manufacture of a medicament for the treatment and/or prevention of amyloidos-related diseases; for the manufacture of a medicament for the treatment and prophylaxis of multiple risk factors known for cardiovascular diseases, such as hypertension and hypertriglyceridemia; for the manufacture of a medicament for prevention of stroke, cerebral or transient ischemic attacks related to atherosclerosis of several arteries; for the manufacturing of a medicament for lowering triglycerides in the blood and/or elevating the HDL cholesterol levels in the serum; and/or for the manufacturing of a medicament for the treatment and/or prevention of the multi-metabolic syndrome termed “metabolic syndrome.” All of those embodiments may also include the use of a fatty acid composition comprising compounds of formula (I) for the manufacture of medicaments as outlined above.

The present disclosure also relates to a method for controlling body weight reduction and for preventing body weight gain, wherein a fatty acid composition comprising at least one compound of formula (I) is administered to a human or an animal.

In another embodiment, the present disclosure relates to a method for the treatment and/or the prevention of obesity or an overweight condition, wherein a fatty acid composition comprising at least one compound of formula (I) is administered to a human or an animal.

In an exemplary embodiment of the present disclosure, the present disclosure relates to a method for the prevention and/or treatment of diabetes mellitus, wherein a fatty acid composition comprising at least one compound of formula (I) is administered to a human or an animal. In at least one embodiment, diabetes mellitus is a type 2 diabetes.

The fatty acid compounds of formula (I) may be prepared from DHA. If the starting material is not pure DHA (i.e. not 100% DHA), the final fatty acid composition will contain a mixture of DHA derivatives, as hereinbefore defined, and an amount of other fatty acids other than DHA, wherein these fatty acids are substituted in the same way, i.e., in the alpha-position, as the novel fatty acid compounds of formula (I). Such embodiments are also included herein.

In another embodiment of the present disclosure, the compounds of formula (I) are prepared from (all-Z)-4,7,10,13,16,19-docosahexaenoic acid (DHA), wherein the DHA is obtained from a vegetable, a microbial, and/or an animal source. In at least one embodiment, DHA is obtained from a marine oil, such as a fish oil.

The fatty acids in the composition may also be obtained from a vegetable, a microbial, or an animal source, or combinations thereof. Thus, the present disclosure also includes a fatty acid composition prepared from a microbial oil.

The present disclosure provides processes for preparing the novel fatty acid compounds of formula (I).

DHA is produced from biological sources like marine, microbial, or vegetable fats. Possible raw materials include mixtures of fatty acids in the triglyceride form where DHA constitutes only a fraction of the fatty acids. Typical DHA concentrations are 40% in microbial fats and 10-25% in marine fats. DHA-containing vegetable fats are under development, and fats with high DHA concentrations are expected in the future.

The first process step comprises the conversion of the triglycerides to free fatty acids or monoesters. Exemplary esters include methyl or ethyl esters, but other esters are possible. In this way, the fatty acids bound together as triglycerides are separated from each other. Several methods of separating DHA from other fatty acids are known. The most common ones include short path distillation (separating the fatty acids by volatility) and urea precipitation (separating the fatty acids by degree of unsaturation). Other methods reported include silver nitrate complexation (separating the fatty acids on degree on unsaturation), esterification reactions catalyzed by fatty acid selective lipases in combination with short path distillation, and countercurrent extraction with supercritical carbon dioxide.

A challenge connected to production of pure DHA is to separate DHA from the other C20-22 highly unsaturated fatty acids present in available sources of DHA. These other fatty acids have properties so similar to DHA that the methods mentioned above may not provide a sufficient degree of separation. For some microbial DHA-containing fats, which have very low levels of C20-22 highly unsaturated fatty acids, short path distillation alone or in combination of other methods mentioned may provide more that 90% purity.

Most DHA containing fats also contain considerable amounts of C20-22 highly unsaturated fatty acids, e.g. EPA (20:5n-3), n-3 DPA (22:5n-3), HPA (21:5n-3) and others. An available method for separating DHA from such fatty acids is preparative high performance liquid chromatography (HPLC), wherein the stationary phase is silica gel or silver nitrate impregnated silica gel, the mobile phase is selected from organic solvents or supercritical carbon dioxide. With this method, DHA with more than 97% purity may be obtained. However, it has to be noted that the production cost increases with concentration. For an example, the production cost for 97% DHA is more than 5 times higher than for 90% DHA.

DHA having a purity of 90%, 95%, and 97% contain small amounts of other fatty acids. As an example, DHA having a purity of 97% contains n-3 DPA (22:5n-3), but also long chain fatty acids, e.g. EPA (20:5n-3), HPA (21:5n-3), and others. However, the other fatty acids will react in a way similar to DHA and provide alpha-substituted derivatives.

Organic synthesis may provide a purification method since DHA and n-6 DPA (and 22:5n-6 which normally is present in very low concentrations) are the only known fatty acids that can provide gamma-lactones by cyclization with the first double bond. Lactonization followed by purification and hydrolysis back to DHA may be a possibility, but it is expected that this pathway is even more expensive than HPLC.

In one embodiment, the compounds of formula (I) where R₁ (or R₂) is a hydrogen are prepared through the following processes (Scheme 1). Suitably adapted, these processes can also be used for preparing compounds of formula (I) where both R₁ and R₂ are, e.g. a C₁-C₇ alkyl group, a benzyl, a halogen, an alkenyl, or an alkynyl.

Compounds represented by the general formula (I) where R₁ is a hydrogen and R₂ denotes a C₁-C₇ alkyl group, a benzyl, a halogen, an alkenyl, an alkynyl are prepared by reacting a DHA ester with a strong non-nucleophilic base, such as lithium diisopropylamine or potassium/sodium hexamethyldisilazide in a solvent such as tetrahydrofuran or diethyl ether at temperatures of −60° C. to −78° C., to provide the ester enolate (process 1).

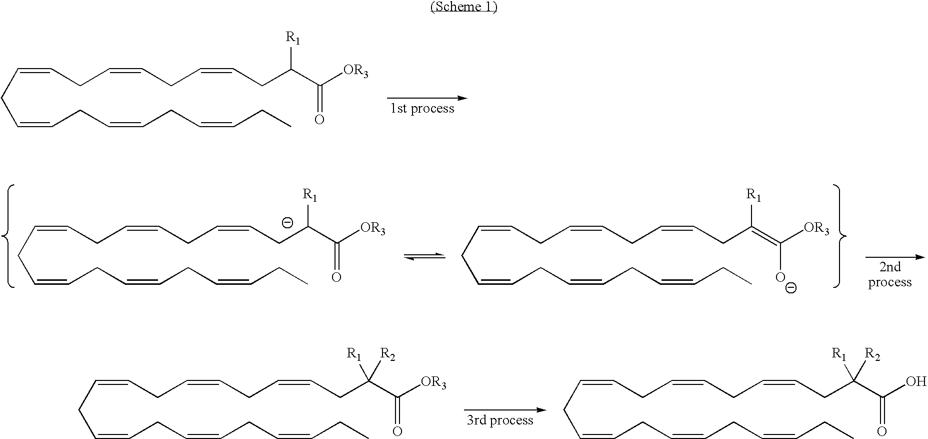

This ester enolate is reacted with an electrophilic reagent, such as an alkylhalide, exemplified by ethyliodine, benzylcloride, an acyl halide exemplified by acetyl chloride, benzoyl bromide, a carboxylic anhydride exemplified by acetic anhydride, or an electrophilic halogenation reagent, exemplified by N-fluorobenzene sulfonimide (NFSI) to provide the monosubstituted derivative (process 2). The ester is further hydrolyzed in a solvent such as ethanol or methanol to the carboxylic acid derivative by the addition of a base, such as lithium/sodium hydroxide in water at temperatures ranging from 15° C. to 40° C.

Claisen condensation of the DHA EE occurs during the treatment of DHA EE with a strong base. This condensation product might possess interesting biologically activity. Thus, in one embodiment of the present disclosure the condensation (intermediate) product mentioned above, as well as the use of this product for treatment and/or prevention of diseases according to the present disclosure, are disclosed.

In a further embodiment, compounds represented by the general formula (I) are synthesized through following processes (Scheme 2).

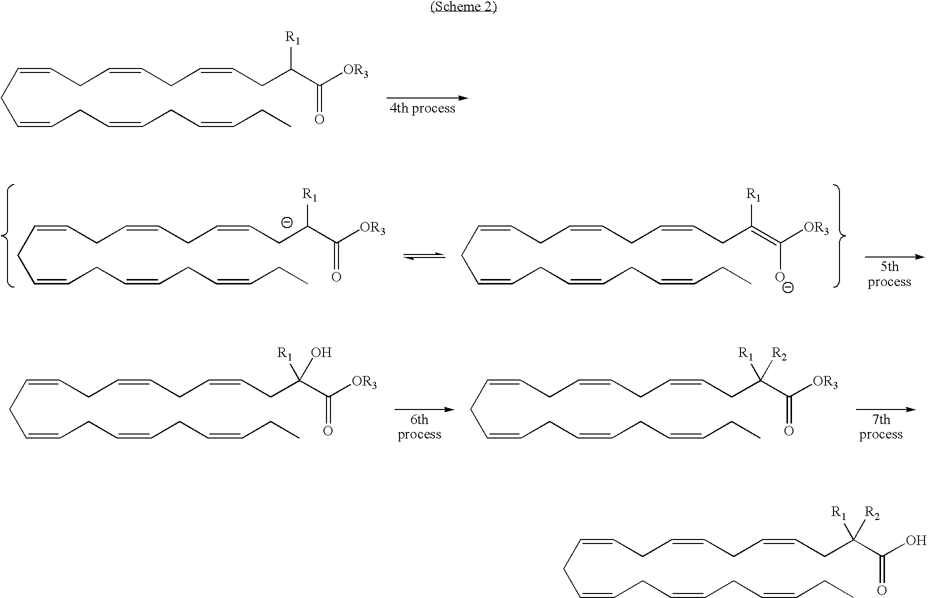

Compounds represented by the general formula (I) where R₁ is a hydrogen and R₂ denotes a hydroxy, an alkoxy group, an acyloxy are prepared by reacting a DHA ester with a strong non-nucleophilic base, such as lithium diisopropylamine or potassium/sodium hexamethyldisilazide in a solvent such as tetrahydrofuran or diethyl ether at temperatures ranging from −60° C. to 78° C., to provide the ester enolate (process 4). This ester etiolate is reacted with an oxygen source like dimethyldioxirane, 2-(phenylsulfonyl)-3-phenyloxaziridine, molecular oxygen with different additives like trimethylphosphite, or different catalysts like a Ni(II) complex to provide alpha-hydroxy DHA ester (process 5). Reaction of the secondary alcohol with a base like sodium hydride in a solvent like THF or DMF generates an alkoxide that is reacted with different electrophilic reagents, such as an alkyliodide, for example: methyl iodide, ethyl iodide, benzylbromide, or an acyl halide, for example: acetyl chloride or benzoyl bromide (process 6). The ester is hydrolyzed in a solvent such as ethanol or methanol to the carboxylic acid derivative by the addition of a base such as lithium/sodium hydroxide in water at temperatures ranging from 15° C. to 40° C. (process 7).

The hydroxy-DHA ester is a useful intermediate for the introduction of other functional groups in the a-position according to the present disclosure. The hydroxyl function can be activated by conversion to a halide or tosylate prior to reaction with different nucleophiles such as ammonia, amines, and thiols. The Mitsunobu reaction is also useful for the conversion of a hydroxyl group into other functional groups. (Mitsunobu, O. Synthesis (1981) 1-28).

Compounds represented by compounds of formula (I) may also be synthesized by combinations of the different processes previously described. The present disclosure includes the processes mentioned above.

The present disclosure further provides a process for the preparation of a pharmaceutical composition comprising mixing at least one compound of formula (I), or a pharmaceutically acceptable salt, solvate, complex, or pro-drug thereof, with at least one pharmaceutically acceptable excipient, diluent, or a carrier.

The enantiomerically pure compounds can be prepared by resolving a racemic compound of formula (I), as hereinbefore defined. The resolution of a compound of formula (I) may be carried out using known resolution procedures, for example by reacting the compound of formula (I) with an enantiomerically pure auxiliary to provide a mixture of diastereomers that can be separated by chromatography. Thereafter the two enantiomers of the compound of formula (I) may be regenerated from the separated diastereomers by conventional means, such as hydrolysis.

There is also a possibility to use stoichiometric chiral auxiliaries to effect an asymmetric introduction of the substituents, as hereinbefore defined, in the α-position of DHA. The use of chiral oxazolidin-2-ones has proved to be a particularly effective method. The enolates derived from chiral N-acyloxazolidines can be quenched with a variety of electrophiles in a highly stereoregulated manner (Ager, D. J. et al. Chem. Rev. (1996) 96:835-876).

### Examples

The present disclosure will now be described in more detail by the following examples, which are not to be constructed as limiting the present disclosure. In the examples the structures were verified by Mass Spectrometry (MS). It should be pointed out that the fatty acid compounds may also be produced from low and medium DHA-containing starting material (i.e. about 40-60 wt % DHA).

There are several common synthetic methods for the preparation of monoglycerides with the fatty acid in the 2-position. One method utilizes esterification of the fatty acid with glycidol in the presence of 1-(3-dimethylaminopropyl)-3-ethylcarbodiimidehydrochloride (EDC) and 4-dimethylaminopyridine (DMAP) to produce a glycidyl derivative. Treatment of the glycidyl derivative with trifluoroacetic anhydride (TFAA) before a trans-esterification reaction produces the monoglyceride (Parkkari et al, Bioorg. Med. Chem. Lett. (2006) 2437).

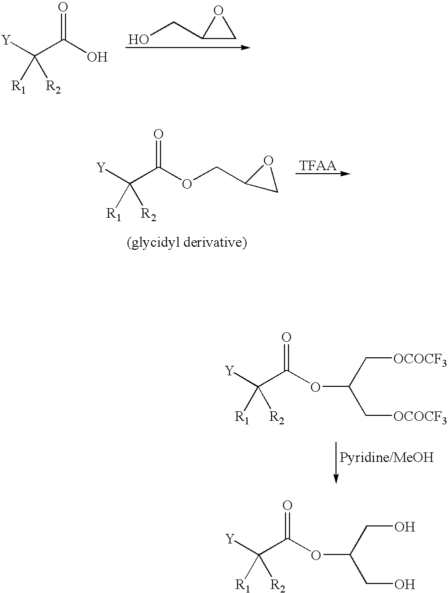

Further common methods for the preparation of mono-, di-, and tri-glycerides of fatty acid derivatives are described in international patent application, PCT/FR02/02831.

It is also possible to use enzymatic processes (lipase reactions) for the transformation of a fatty acids to a mono-, di-, or triglyceride. A 1,3-regiospecific lipase from the fungus Mucor miehei can be used to produce triglycerides or diglycerides from polyunsaturated fatty acids and glycerol. A different lipase, the non-regiospecific yeast lipase from Candida antartica is highly efficient in generating triglycerides from polyunsaturated fatty acids (Haraldsson, Pharmazie (2000) 3).

### Synthesis Protocols

Precursors to the compounds of formula (I), such as the free fatty acids and/or ethyl ester derivatives, can be prepared as described in WO 2006/117664.

Preparation of α-ethyl DHA EE (PRB-2)

Butyllithium (440 ml, 0.67 mol, 1.6 M in hexane) was added dropwise to a stirred solution of diisopropylamine (111 ml, 0.78 mol) in dry THF (750 ml) under N₂ at 0° C. The resulting solution was stirred at −78° C. for 45 min. before dropwise addition of DHA EE (200 g, 0.56 mol) in dry THF (1.61). The addition of the ester was complete in 4 hours. The dark-green solution was stirred at −78° C. for 30 min. before EtI (65 ml, 0.81 mol) was added. The solution was allowed to reach −40° C. before an additional amount of EtI (5 ml, 0.06 mol) was added, and finally reach −15° C. (during 3 hours from −78° C.) before the mixture was poured into water and extracted with hexane (2×). The combined organic phases were washed with 1 M HCl, water, dried (Na₂SO₄), filtered and evaporated in vacuo. The product was purified by flash chromatography on silica gel eluting with heptane/EtOAc (99:1 followed by 50:1) to give 42.2 g (20%) of the titled compound as a yellow oil;

¹H-NMR (200 MHz; CDCl₃) δ 0.8-1.0 (m, 6H), 1.2-1.4 (m, 411), 1.5-1.7 (m, 2H), 2.12 (m, 2H), 2.3-2.5 (m, 2H), 2.8-3.0 (m, 10H), 4.18 (t, J 7.1 Hz, 211), 5.3-5.6 (m, 12H);

MS (electrospray); 407 [M+Na].

### Preparation of PRB-2 MG

PRB-A

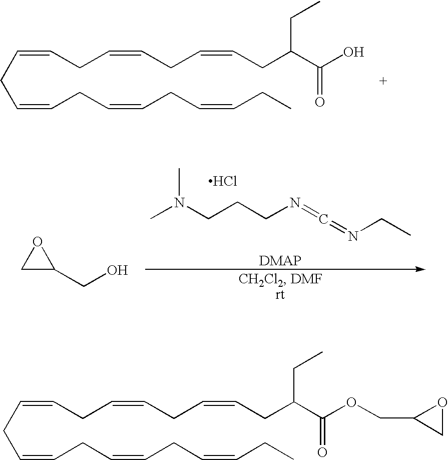

A solution of PRB-2 FA ((all-Z)4,7,10,13,16,19-2-ethyldocosahexaenoic acid) (5.081 g, 14.25 mmol), glycidol (0.63 ml, 9.5 mmol), N-Ethyl-N′-(3-dimethylaminopropyl)carbodiimide hydrochloride (2.745 g, 14.3 mmol) and DMAP (1.752 g, 14.3 mmol) in CH₂Cl₂, 60 ml, was stirred for 113 hrs under N₂-atmosphere at room temperature before evaporation in vacuo. Flash chromatography on silica gel eluting with heptane:EtOAc (95:5) yielded 1.396 g (36%) of the desired product as a yellow liquid.

¹H NMR (300 MHz, CDCl₃) δ 0.89 (t, J=7.5 Hz, 3H), 0.95 (t, J=7.6 Hz, 3H), 1.55-1.64 (m, 2H), 2.05 (quint, J=7.4 Hz, 2H), 2.25-2.37 (m, 3H), 2.60-2.63 (m, 1H), 2.77-2.84 (m, 11H), 3.15-3.19 (m, 1H), 3.89 (ddd, J=12.3 Hz, 6.3 Hz, 3.8 Hz, 1H), 4.39 (dd, J=12.3 Hz, 3.1 Hz, 1H), 5.27-5.42 (m, 12H)

¹³C NMR (75 MHz, CDCl₃) δ 11.7, 14.3, 20.5, 24.8, 24.9, 25.5, 25.6 (2 C), 29.47, 29.53, 44.6, 47.07, 49.4, 64.6, 64.7, 126.5, 127.0, 127.8, 128.01, 128.04, 128.08, 128.18, 128.21, 128.5, 129.9, 132.0, 175.3

MS (electrospray); 435 [M+Na]⁺

PRB-B

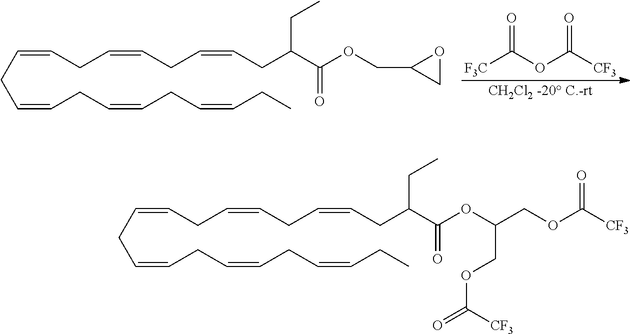

A solution of PRB-A (1.368 g, 3.31 mmol) in dry CH₂Cl₂, 15 ml, was cooled to −20° C. under N₂-atmosphere before a solution of trifluoroacetic acid anhydride (TFAA) (1.85 ml, 13.3 mmol) in dry CH₂Cl₂, 15 ml, was added portion wise. The cooling bath was removed and the mixture was stirred for 1 hr. The solvent and unreacted TFAA was evaporated in vacuo and the residue was dissolved in toluene 30 ml and passed through a silica gel pad (30 g) eluting with toluene, 700 ml. Evaporation in vacuo yielded 1.21 g (59%) of the crude 1,3-bis(trifluoroacetyl)-2-PRB-2.

¹H NMR (300 MHz, CDCl₃) δ 0.87 (t, J=7.4 Hz, 3H), 0.95 (t, J=7.5 Hz, 3H), 1.60 (m, 2H), 2.05 (quint, J=7.3 Hz, 2H), 2.21-2.38 (m, 3H), 2.79-2.83 (m, 10H), 4.39-4.48 (m, 2H), 4.56-4.63 (m, 2H), 5.29-5.42 (m, 13H)

¹³C NMR (75 MHz, CDCl₃) δ 11.5, 14.2, 20.5, 24.8, 25.5, 25.6 (2 C), 29.4, 47.0, 64.8, 66.9, 125.9, 127.0, 127.76, 127.83, 128.0, 128.3, 128.4, 128.6, 130.3, 132.0, 174.6 (unfortunately too few scans to see the COCF₃ signals)

PRB-2 MG

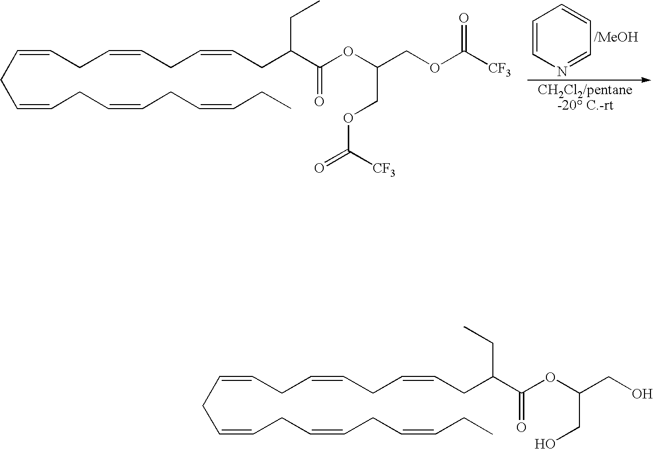

A solution of PRB-B in pentane/CH₂Cl₂ (2:1), 20 ml, was cooled to −20° C. under N₂-atmosphere before drop wise addition of pyridine (1.6 ml, 19.8 mmol) and MeOH (1.2 ml, 29.6 mmol). The cooling bath was removed and the mixture was stirred for 4 hrs before evaporation in vacuo. Flash chromatography on silica gel eluting with heptane-heptane:EtOAc 1:1 yielded 670 mg (80%) of PRB-2 MG as a light yellow oil.

¹H NMR (300 MHz, CDCl₃) δ 0.90 (t, J=7.4 Hz, 3H), 0.92 (t, J=7.5 Hz, 3H), 1.50-1.70 (m, 2H), 2.05 (quint., J=7.3 Hz, 2H), 2.19-2.31 (m, 3H), 2.32-2.43 (m, 2H), 2.74-2.87 (m, 10 H), 3.77 (m, 4H), 4.91 (m, 1H), 5.11-5.46 (m, 12H)

¹³C NMR (75 MHz, CDCl₃) δ 11.7, 14.2, 20.5, 25.0, 25.5, 25.6 (2C), 29.7, 47.3, 62.2, 75.0, 126.6, 126.9, 127.8, 128.0, 128.2, 128.3, 128.5, 130.0, 132.0, 175.9 (5 signals hidden)

MS (electrospray); 453 [M+Na]⁺

HPLC; 98%

### Test Example 1

### Demonstration of Effects on Lipid Metabolism in vivo

An exemplary composition comprising PRB-2 MG was tested in an animal model as described below.

Because 3-thia fatty acids such as tetradecylthioacetic acid (TTA) are known to decrease serum triglycerides, cholesterol, and free fatty acid levels in animal models (Journal of Lipid Research, 1999, 2099) this substance was used as a positive control in our animal study.

### Mice

Female heterozygous APOE*3Leiden mice were used and housed during the experiment in macrolon cages (three or four mice per cage) in clean-conventional animal rooms (relative humidity 50-60%, temperature ˜21° C., light cycle 7 am to 7 pm). Individual animals were marked by ear punch-holes. Mice were supplied with food and acidified tap water ad libitum.

### Diets

The mice received a semi-synthetic modified Western-type diet (WTD) as described by Nishina et al (J Lipid Res 1990; 31: 859), containing cholesterol (0.25% w/w, final concentration) and 15% cacao butter.

### Drug Administration

PRB-2 MG and TTA were administered orally as admix to the Western-type diet in 0.3 mmol/kg bw/day. The lyophilized diet chunks were stored in vacuum bags in the dark in an alarm-secured −20° C. room. The diets on the cages of the mice were changed twice a week.

### Study Design

APOE*3 Leiden mice were put on a semi-synthetic Western-type diet. After a 4-week run-in period low-responder mice was removed form the study, and the remaining mice were sub-divided into groups of 10 mice each, matched for plasma cholesterol, triglycerides, free fatty acids, and age (t=0).

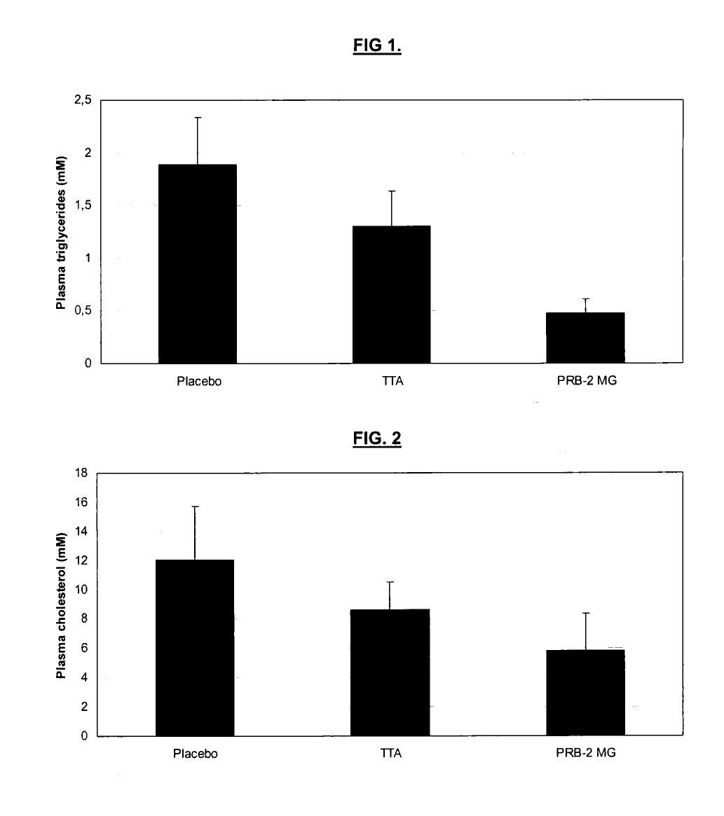

After t=0 and 4 weeks, body weight and food intake were measured and blood samples were taken after a 4-hour fasting period to measure plasma cholesterol and triglycerides, and lipoprotein profile. After 4 weeks, all animals were sacrificed. The results are shown in FIGS. 1 and 2. As evident from those results, it was shown that an exemplary compound of the present disclosure had lipid lowering effects that were better than those effects for TTA, the positive control. From the lipoprotein profiles, it is evident that the lipid lowering effects are related to non-HDL particles.

Data for ApoE mice are reported in FIGS. 1 and 2 for triglycerides and cholesterol, respectively.

### Test Example 2

### Evaluation of PPAR Activation in-vitro

### Method

The assay was carried out in vitro in three stable reporter cell lines, PPARα, PPARδ, or PPARγ, expressing respectively a chimeric protein containing the ligand binding domain (LBD) of human PPARα, human PPARδ, or human PPARγ fused to the yeast transactivator GAL4 DNA binding domain (DBD).

The luciferase (Luc) reporter gene is driven by a pentamer of the GAL4 recognition sequence in front of a β-globin promoter. The use of GAL4-PPARα, GAL4-PPARδ and GAL4-PPARγ chimeric receptors allows for elimination of background activity from endogenous receptors and quantitation of relative activity across the three PPAR subtypes with the same reporter gene.

The PPAR activity of PRB-2 MG (10 μM) was determined by comparison to known drug references (1 μM GW7647 for PPARα, 1 μM L-165041 for PPARδ and 1 μM BRL49653 for PPARγ) and a negative control (0.1% DMSO). The results are presented as percentage activity compared to positive control (set to 100%).

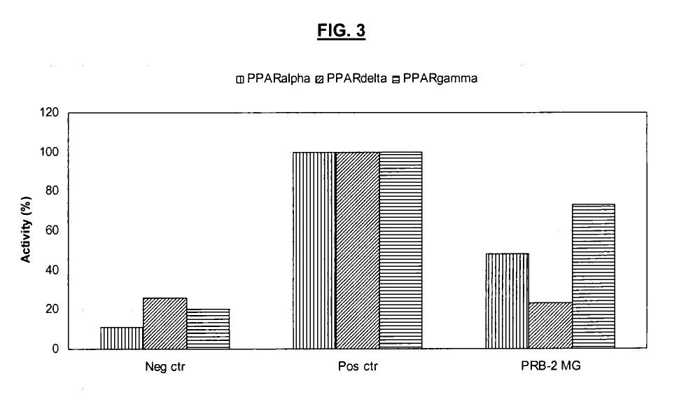

The PPAR activation data is shown in FIG. 3.

1. A compound of formula (I): 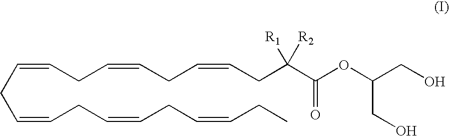
wherein
R₁ and R₂ are the same or different and are chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, an alkylsulfinyl group, an alkylsulfonyl group, an amino group, and an alkylamino group;
or any pharmaceutically acceptable salt, solvate, complex, or pro-drug thereof.

2. The compound according to claim 1, wherein the alkyl group is chosen from methyl, ethyl, n-propyl, isopropyl, n-butyl, iso-butyl, sec-butyl, n-hexyl, and benzyl.

3. The compound according to claim 1, wherein the alkoxy group is chosen from methoxy, ethoxy, propoxy, isopropoxy, sec-butoxy, phenoxy, benzyloxy, OCH₂CF₃, and OCH₂CH₂OCH₃.

4. The compound according to claim 1, wherein R₁ and R₂ are chosen from an alkyl group.

5. The compound according to claim 1, wherein R₁ and R₂ are chosen from a C₁-C₇ alkyl group and a C₁-C₇ alkoxy group.

6. The compound according to claim 5, wherein the C₁-C₇ alkyl group is chosen from methyl, ethyl, and propyl; and the C₁-C₇ alkoxy group is chosen from methoxy and ethoxy.

7. The compound according to claim 1, wherein R₁ and R₂ are different.

8. The compound according to claim 7, wherein the compound is in racemic form.

9. The compound according to claim 7, wherein the compound is in the form of its R stereoisomer.

10. The compound according to claim 7, wherein the compound is in the form of its S stereoisomer.

11. The compound according to claim 1, wherein one of R₁ and R₂ is chosen from C₁-C₇ alkyl groups and C₁-C₇ alkoxy groups, and the other one is a hydrogen atom.

12. The compound according to claim 11, wherein one of R₁ and R₂ is chosen from methyl, ethyl, and propyl.

13. The compound according to claim 11, wherein one of R₁ and R₂ is chosen from methoxy, ethoxy, and propoxy.

14. The compound according to claim 11, wherein the alkyl group is ethyl and is a compound of formula (Ia) 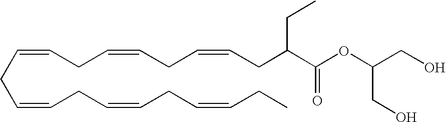
(Ia) (all-Z)-1,3-dihydroxypropan-2-yl 2-ethyl-docosa-4,7,10,13,16,19-hexaenoate.

15. The compound according to claim 14, wherein the compound is in racemic form.

16. The compound according to claim 14, wherein the compound is in the form of its R stereoisomer.

17. The compound according to claim 14, wherein the compound is in the form of its S stereoisomer.

18. (canceled)

19. A process for the manufacture of a compound according to claim 1 comprising
a) reacting (all-Z)-4,7,10,13,16,19-docosahexaenoic acid (DHA) with a strong non-nucleophilic based;
b) reacting the enolate formed in step a) with an electrophilic reagent; and
c) reacting the product of step c) with either glycidol or a lipase to obtain the compound.

20. (canceled)

21. The process according to claim 19, wherein the DHA originated from a marine oil.

22. A pharmaceutical composition comprising a compound according to claim 1.

23. A pharmaceutical composition comprising a compound of formula (Ia) 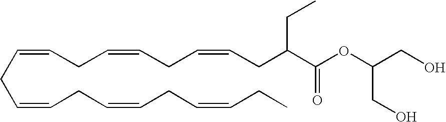
(Ia) (all-Z)-1,3-dihydroxypropan-2-yl 2-ethyl-docosa-4,7,10,13,16,19-hexaenoate.

24. The pharmaceutical composition according to claim 22, further comprising a pharmaceutically acceptable carrier.

25. The pharmaceutical composition according to claim 24, wherein said pharmaceutical composition is formulated for oral administration.

26. The pharmaceutical composition according to claim 25, wherein said pharmaceutical composition is in a form chosen from capsule form, tablet form, and sachet form.

27. The pharmaceutical composition according to claim 22, formulated to provide a daily dosage ranging from 10 mg to 10 g of the compound.

28. The pharmaceutical composition according to claim 27, formulated to provide a daily dosage ranging from 100 mg to 1 g of the compound.

29-38. (canceled)

39. A method for the treatment and/or the prevention of peripheral insulin resistance and/or a diabetic condition comprising administering a pharmaceutically effective amount of a compound according to claim 1 to a human or an animal in need thereof.

40. A method for reduction of plasma insulin, blood glucose, and/or serum triglycerides comprising administering a pharmaceutically effective amount of a compound according to claim 1 to a human or an animal in need thereof.

41. A method for the treatment and/or the prevention of type 2 diabetes comprising administering a pharmaceutically effective amount of a compound according to claim 1 to a human or an animal in need thereof.

42. A method for the prevention and/or treatment of elevated triglyceride levels, and/or non-HDL cholesterol, LDL cholesterol, and VLDL cholesterol levels comprising administering a pharmaceutically effective amount of a compound according to claim 1 a human or an animal in need thereof.

43. A method for the prevention and/or treatment of a hyperlipidemic condition comprising administering a pharmaceutically effective amount of a compound according to claim 1 to a human or an animal in need thereof.

44. The method according to claim 43, wherein the hyperlipidemic condition is hypertriglyceridemia (HTG).

45. A method for the treatment and/or the prevention of obesity or an overweight condition comprising administering a pharmaceutically effective amount of a compound according to claim 1 to a human or an animal in need thereof.

46. A method for reduction of body weight and/or for preventing body weight gain comprising administering a pharmaceutically effective amount of a compound according to claim 1 to a human or an animal in need thereof.

47. A method for treatment of insulin resistance, hyperlipidemia and/or obesity or an overweight condition comprising administering a pharmaceutically effective amount of a compound according to claim 1 to a human or an animal in need thereof.

48. A method for the treatment and/or the prevention of an inflammatory disease or condition comprising administering a pharmaceutically effective amount of a compound according to claim 1 to a human or an animal in need thereof.

49. The method according to any claim 39, wherein the compound is administered orally.

50. A method for preparing a compound of formula (Ia) 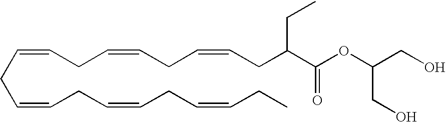
(Ia) (all-Z)-1,3-dihydroxypropan-2-yl 2-ethyl-docosa-4,7,10,13,16,19-hexaenoate comprising:
a) reacting (all-Z)-4,7,10,13,16,19-2-ethyldocosahexaenoic acid with glycidol in the presence of a coupling reagent;
b) reacting the product from step a) with trifluoroacetic acid anhydride;
c) reacting the product from step b) with pyridine;
d) isolating the product from step c) and optionally purifying.

51. The method according to claim 50, wherein the product from any one of steps a), b), or c) is optionally isolated and/or purified.
